(19) United States
(12) Patent Application Publication
(10) Pub. No.: US 2022/0036891 A1
(43) Pub. Date: Feb. 3, 2022

(54) CUSTOMIZING A POLICY OF AN INPUT/OUTPUT DEVICE IN RESPONSE TO USER CONSTRAINTS

(71) Applicant: Intuition Robotics, Ltd, Ramat-Gan (IL)

(72) Inventors: Shay ZWEIG, Harel (IL); David EDERY, Netanya (IL); Itai MENDELSOHN, Tel Aviv (IL); Roy AMIR, Mikhmoret (IL); Dor SKULER, Oranit (IL)

(73) Assignee: Intuition Robotics, Ltd., Ramat-Gan (IL)

(21) Appl. No.: 17/388,654

(22) Filed: Jul. 29, 2021

Related U.S. Application Data
(60) Provisional application No. 63/058,770, filed on Jul. 30, 2020.

(51) Int. Cl. G10L 15/18; G10L 15/22

(52) U.S. Cl. CPC G10L 15/1822; G16H 20/30; G10L 15/22

(57) ABSTRACT

A method and system for generating a modified input/output (I/O) device policy in response to constraints of a user are provided. The method includes determining at least one constraint of a user using the I/O device policy in response to a request to perform a plan defined in the policy, wherein the at least one constraint is determined based on a first dataset collected from different electronic sources connected to the I/O device; generating a first plan customizing the policy based on the at least one determined constraint, wherein the first plan demonstrates a high acceptance score, wherein the acceptance score defines a probability that the user accepts and fulfills the first plan; and executing the first plan defined in the customized policy, wherein executing the first plan further includes causing the I/O device to activate an action that complies with the at least one determined constraint.

### CROSS-REFERENCE TO RELATED APPLICATIONS

This application claims the benefit of U.S. Provisional Application No. 63/058,770 filed on Jul. 30, 2020, the contents of which are hereby incorporated by reference.

### TECHNICAL FIELD

The disclosure generally relates to input/output devices, and more specifically to a system and method for generating a customized plan for a user of using the input/output device.

### BACKGROUND

As manufacturers improve the functionality of devices such as vehicles, computers, mobile phones, appliances, and the like, through the addition of digital features, manufacturers and end-users may desire enhanced device functionalities. The manufacturers, as well as the relevant end-users, may desire digital features which improve user experiences, interactions, and features which provide for greater connectivity. Certain manufacturers may include device-specific features, such as setup wizards and virtual assistants, to improve device utility and functionality. Further, certain software packages may be added to devices, either at the point of manufacture, or by a user after purchase, to improve device functionality. Such software packages may provide functionalities including, as examples, a computer system's voice control, facial recognition, biometric authentication, and the like.

While the features and functionalities described hereinabove provide for certain enhancements to a user's experience when interacting with a device, the same features and functionalities, as may be added to a device by a user or manufacturer, fail to include certain aspects which may allow for a further-enhanced user experience. First, certain currently-implemented digital assistants and other user experience features may fail to provide for an adaptive adjustment of the operation of the assistant or feature based on immediate or planned users' constraints. As such, users may not benefit from policies or actions performed or suggested by the digital assistants, and eventual abandon the usage of such devices.

It would therefore be advantageous to provide a solution that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for generating a modified input/output (I/O) device policy in response to constraints of a user, comprising: determining at least one constraint of a user using the I/O device policy in response to a request to perform a plan defined in the policy, wherein the at least one constraint is determined based on a first dataset collected from different electronic sources connected to the I/O device; generating a first plan customizing the policy based on the at least one determined constraint, wherein the first plan demonstrates a high acceptance score, wherein the acceptance score defines a probability that the user accepts and fulfills the first plan; and executing the first plan defined in the customized policy, wherein executing the first plan further includes causing the I/O device to activate an action that complies with the at least one determined constraint.

Certain embodiments disclosed herein a system for generating a modified input/output (I/O) device policy in response to constraints of a user, comprising: a processing circuitry; and a memory, the memory containing instructions that, when executed by the processing circuitry, configure the system to: determine at least one constraint of a user using the I/O device policy in response to a request to perform a plan defined in the policy, wherein the at least one constraint is determined based on a first dataset collected from different electronic sources connected to the I/O device;

generate a first plan customizing the policy based on the at least one determined constraint, wherein the first plan demonstrates a high acceptance score, wherein the acceptance score defines a probability that the user accepts and fulfills the first plan; and execute the first plan defined in the customized policy, wherein executing the first plan further includes causing the I/O device to activate an action that complies with the at least one determined constraint.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a network diagram depicting a network diagram utilized to describe the various embodiments of customizing policies of an input/output device policy based on users' constraints.

FIG. 2 is a block diagram of a controller designed according to an embodiment.

FIG. 3 is a flowchart illustrating a method for customizing policies of an input/output device policy based on users' constraints.

FIG. 4 is a flowchart of a method for further customizing a policy in response to rejection of a customized policy, according to an embodiment.

### DETAILED DESCRIPTION

The embodiments disclosed by the disclosure are only examples of the many possible advantageous uses and implementations of the innovative teachings presented herein. In general, statements made in the specification of the present application do not necessarily limit any of the various claimed disclosures. Moreover, some statements may apply to some inventive features but not to others. In general, unless otherwise indicated, singular elements may be in plural and vice versa with no loss of generality. In the drawings, like numerals refer to like parts through several views.

The embodiments disclosed herein relate to the analysis of sensor data, as may relate to a user, the conditions of a user's environment, external events, and the like, as well as the application of such analyzed data to the generation, customization, and execution of various digital assistant programs, routines, services, and the like. The disclosed embodiments provide for reduced processing time and, thus, improved computational efficiency in the modification and execution of digital assistant routines; in particular, where the modification and execution of such routines include analysis of one or more attributes or data features relating to user constraints. Such constraints may include, for example, routine, future, planned, and/or unplanned activities of the user, user's requests, and restrictions related thereto, and the like.

By collecting and analyzing data related to a user, an initial policy defined for the I/O device operating the digital assistant is modified. The initial policy defines plans or actions to be executed by the digital assistant for a given current state of a user. A modified or customized policy, which suits the preferences and the needs of a user while factoring the user constraints, is generated. The modified policy is used for controlling the execution of plans, in order to adjust the initial plan(s) to a user without negatively effecting the user experience when the user constraints are factored. The plans executed by the digital assistant include causing outputs via one or more input/output (I/O) devices.

The embodiments disclosed herein further provide specific advantages in the solution of digital assistant customization problems. Enjoying the performance and experience of electronic gadgets (such as digital assistant) is key for the success of such products. While users may be able to manually change plans or policies with respect to known user constraints, such a manual process is time-consuming, requires extensive communication between a user and the digital assistant, and is, ultimately a subjective process.

Moreover, the embodiments disclosed herein provide for objective modification of a policy executed by the digital assistant to address user constraints which suit the preferences of a user. Moreover, historical data related to a user's preferences with respect to outputs by I/O device may be utilized to generate a modified policy for a user with respect to the I/O device.

Additionally, in accordance with various disclosed embodiments, a customized policy is generated based on an initial policy. As the customization related to the user constrains are performed with respect to the initial policy, this limits the number of modifications to the initial policy and improves the accuracy of the customized policy by using the most up-to-date policies of an individual user when the modified policy is about to be used to execute one or more plans. This accuracy, in turn, improves efficiency of the I/O device(s) being controlled, by avoiding unsuitable outputs which would, in turn, prompt a user to manually reconfigure the I/O device.

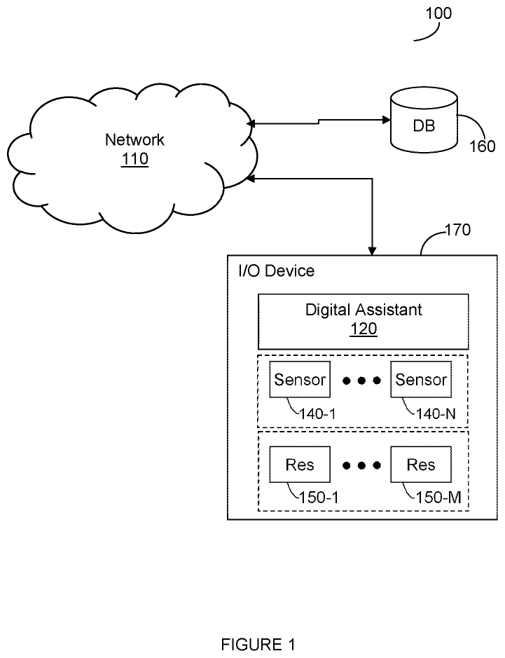

FIG. 1 is an example network diagram 100 utilized to describe the various disclosed embodiments. The network diagram 100 includes an input/output (I/O) device 170 operating a digital assistant 120. In some embodiments, the digital assistant 120 is further connected to a network 110 to allow some processing of a remote server (e.g., a cloud server). The network 110 may provide for communication between the elements shown in the network diagram 100. The network 110 may be, but is not limited to, a local area network (LAN), a wide area network (WAN), a metro area network (MAN), the Internet, a wireless, cellular, or wired network, and the like, and any combination thereof.

In an embodiment, the digital assistant 120 may be connected to, or implemented on, the I/O device 170. The I/O device 170 may be, for example and without limitation, a robot, a social robot, a service robot, a smart TV, a smartphone, a wearable device, a vehicle, a computer, a smart appliance, and the like.

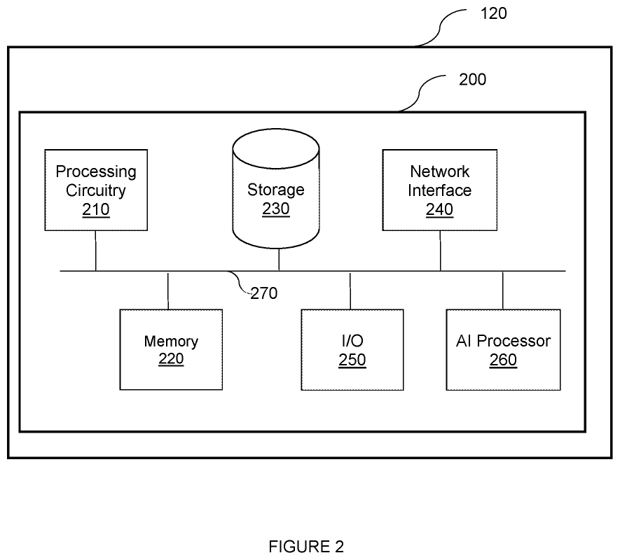

The digital assistant 120 may be realized in software, firmware, hardware, combination thereof. An example block diagram of the digital assistant 120 is provided in FIG. 2. The digital assistant 120 is configured to process sensor data collected by one or more sensors, 140-1 to 140-N, where N is an integer equal to or greater than 1 (hereinafter referred to as “sensor” 140 or “sensors” 140 for simplicity) and one or more resources 150-1 to 150-M, where M is an integer equal to or greater than 1 (hereinafter referred to as “resource” 150 or “resources” 150 for simplicity). The resources 150 may include, for example, electro-mechanical elements, display units, speakers, and the like. In an embodiment, the resources 150 may include sensors 140 as well. The sensors 140 and the resources 150 are included in the I/O device 170.

The sensors 140 may include input devices, such as various sensors, detectors, microphones, touch sensors, movement detectors, cameras, and the like. Any of the sensors 140 may be, but are not necessarily, communicatively, or otherwise connected to the digital assistant 120 (such connection is not illustrated in FIG. 1 for the sake of simplicity and without limitation on the disclosed embodiments). The sensors 140 may be configured to sense signals received from a user interacting with the I/O device 170 or the digital assistant 120, signals received from the environment surrounding the user, and the like. In an embodiment, the sensors 140 may be implemented as virtual sensors that receive inputs from online services, e.g., the weather forecast, a user's calendar, and the like.

In an embodiment, the network diagram 100 further includes a database (DB) 160. The database 160 may be stored within the I/O device 170 (e.g., within a storage device not shown), or may be separate from the I/O device 170 and connected thereto via the network 110. The database 160 may be utilized for storing, for example, historical data about one or more users, users' preferences and related policies, and the like, as well as any combination thereof.

According to some examples, the digital assistant 120 is configured to customize an initial policy of the assistant 120 in response to constraints of a user (of the assistant 120. User's constraints may include, for example, routine, future, planned, and/or unplanned activities of the user, user's requests, and restrictions related thereto, and so on. To this end, the digital assistant 120 is configured to collect a dataset from one or more sources. A source is a source of information, such as for example, a user's electronic calendar, social media account, sensors, navigation program, database, and so on. The source may be realized as software, hardware and a combination thereof. Further, an interface, such as API, may be provided to such source.

The first analysis is performed using the dataset to determine whether there are any identified constraints for fulfilling the request of the user, perform a second analysis of the dataset, upon determination that at least one limitation is identified, in order to determine a first plan to be generated by the digital assistant having a highest probability score (“acceptance score”) to be accepted by the user; and, present the first customized plan to the user. The operation of the digital assistant 120 is further discussed below.

FIG. 2 is an example block diagram of a controller 200 acting as a hardware layer of a digital assistant 120, according to an embodiment. The controller 200 includes a processing circuitry 210 that is configured to receive data, analyze data, generate outputs, and the like, as further described hereinbelow. The processing circuitry 210 may be realized as one or more hardware logic components and circuits. For example, and without limitation, illustrative types of hardware logic components that can be used include field programmable gate arrays (FPGAs), application-specific integrated circuits (ASICs), application-specific standard products (ASSPs), system-on-a-chip systems (SOCs), general-purpose microprocessors, microcontrollers, digital signal processors (DSPs), and the like, or any other hardware logic components that can perform calculations or other manipulations of information.

The controller 200 further includes a memory 220. The memory 220 may contain therein instructions that, when executed by the processing circuitry 210, cause the controller 200 to execute actions as further described hereinbelow. The memory 220 may further store therein information, e.g., data associated with one or more users, historical data about one or more users, users' preferences and related policies, and the like.

The storage 230 may be magnetic storage, optical storage, and the like, and may be realized, for example, as a flash memory or other memory technology, or any other medium which can be used to store the desired information.

In an embodiment, the controller 200 includes a network interface 240 that is configured to connect to a network, e.g., the network 110 of FIG. 1. The network interface 240 may include, but is not limited to, a wired interface (e.g., an Ethernet port) or a wireless port (e.g., an 802.11 compliant Wi-Fi card), configured to connect to a network (not shown).

The controller 200 further includes an input/output (I/O) interface 250 configured to control the resources 150 (shown in FIG. 1). In an embodiment, the I/O interface 250 is configured to receive one or more signals captured by the sensors 140 (see FIG. 1) of the digital assistant 120 and to send such signals to the processing circuitry 210 for analysis. According to an embodiment, the I/O interface 250 is configured to analyze the signals captured by the sensors 140, detectors, and the like. According to a further embodiment, the I/O interface 250 is configured to send one or more commands to one or more of the resources 150 for executing one or more plans (e.g., actions) of the digital assistant 120, as further discussed hereinbelow. A plan may include, for example, suggesting that the user play Jazz music via a specific streaming service, suggesting initiation of a navigation plan to a specific address through a selected navigation app, and the like. A plan may also include a set of attributes such as a certain, approach (e.g., cynical, kind, etc.), tone, and the like, to be used for presenting a certain reminder, suggestion, etc. to the user. The plan and its specific attributes may be selected based on user's preferences, historical information, behavioral patterns, etc. According to a further embodiment, the components of the controller 200 are connected via a bus 270.

In some configurations, the controller 200 may further include an artificial intelligence (AI) processor 260. The AI processor 260 may be realized as one or more hardware logic components and circuits, including graphics processing units (GPUs), tensor processing units (TPUs), neural processing units, vision processing units (VPU), reconfigurable field-programmable gate arrays (FPGA), and the like. The AI processor 260 is configured to perform, for example, machine learning based on sensory inputs received from the I/O interface 250, where the I/O interface 250 receives input data, such as sensory inputs, from the sensors 140.

In an embodiment, a policy (including the initial policy) may include a set of behavior rules to be used by the I/O device 170 when interacting with a specific user in particular circumstances. The set of behavior rules may include specific guidelines indicating which plan is to be executed by the I/O device 170, and in which manner, when a specific user is identified in a particular situation. In an embodiment, the policy may be learned and developed through time based on, for example, user behavior, user patterns, user feedback to actions performed by the digital assistant 120, and the like. The initial policy may be preconfigured or learned from user behavior, using reinforcement learning techniques.

In an embodiment, the controller 200 is configured to generate and present an initial policy for a user of the digital assistant 120 to perform an action. The controller 200 may previously determine and generate the recommendation based on at least one predetermined goal of the user. A goal may be for example, improving the user's physical health, mental health, cognitive activity, social relationships, and so on. The initial policy may include actions or recommendations such as, for example and without limitation, suggesting the user to start exercising, suggesting the user to go out for a walk, reminding the user to call a close friend, reminding the user to take medications on time, and so on.

According to one embodiment, a predetermined goal may be associated with a score allowing to determine whether a goal was achieved or not and what is the achievement status of each goal. For example, each goal may have a score from 0 to 10, when 0 is the lowest value and 10 is the highest value. A score of 10 means that the goal was achieved and a value of 0 to + means that the user still needs to accomplish certain activities, missions, and so on, to achieve a certain goal.

In an embodiment, the controller 200 may collect and analyze information (historical and real-time data) prior to determining which recommendation or action in the policy should be presented. For example, the controller 200 may collect and analyze information indicating that the user did not do any physical activity today (although the user usually goes out for a walk every day at 10 am), that the current time is 6:30 pm, that the user usually eats at 7:30 pm, and so on.

In an embodiment, based on analysis of the collected information the controller 200 may generate a recommendation for the user encouraging the user to go out for a walk before dinner. It should be noted that the analysis of the collected information may be achieved by one or more computer vision techniques, audio signal processing techniques, machine learning techniques, rules-based processing, an expert system trained by a machine learning model, and the like.

In an embodiment, the controller 200 is configured to collect feedback data from the user regarding the recommendation. The first feedback data includes real-time data that may be sensed by one or more sensors (e.g., the sensors 140). The first feedback data may include words that the user says, facial expressions, gestures, and so on. For example, after the digital assistant 120 generates a suggestion to the user to exercise as the user has not yet completed their daily workout, the user may say: “I am tired, I think I will give myself a break today”. This would be considered as rejection of the plan.

In an embodiment, the controller 200 may identify and compute more than one item of the feedback data. That is, the user's tone may be determined as well as the user's facial expression and not just the words the user says. By using more than one item of the feedback data, the user's feedback may be determined more accurately, and as such determining a more accurate plan to be executed by the digital assistant 120, as further discussed herein below.

In an embodiment, the controller 200 is further configured to analyze the collected feedback data. The analysis of the first feedback data may be achieved by one or more computer vision techniques, audio signal processing techniques, machine learning techniques, and the like. For example, the first feedback data as sensed by one or more sensors (e.g., the sensors 140) is fed into a designated algorithm, utilized to determine what is the user's feedback (e.g., reaction). It should be noted that the user feedback data, when analyzed, allows to determine whether the user accepts or rejects the recommendation of the digital assistant, the intensity level of a rejection, and so on.

In an embodiment, upon determination that the user rejects the recommendation, the controller 200 may collect a dataset from one or more data sources (user's electronic calendar, social media, sensors, database, etc.). The dataset may include, for example, data regarding the user's patterns and habits, user's schedule, real-time data, data that is gathered from the user's social media accounts, data that is gathered from websites and/or databases (e.g., weather forecast), and so on.

In an embodiment, at least a portion of the dataset is collected by at least one sensor (e.g., the sensors 140) that is communicatively connected to the digital assistant 120. The dataset may include real-time data regarding the user and the environment in a predetermined proximity to the user. The predetermined proximity may be for example, 10 meters from the digital assistant, 6 meters from the user, and the like.

It should be noted that upon determination that the user accepts the recommendation of the digital assistant 120, the controller 200 may be configured to monitor the user's activity and update the achievement status of one or more predetermined goals. The achievement status may be on a scale from 1 to 10, allowing to track the level of fulfillment of each goal per day, week, month, and so on. For example, if the achievement status of the physical activity goal is 6 out of 10 and the user just accepted the recommendation of practicing yoga for 30 minutes (and practiced yoga for 30 minutes), the achievement status may be updated respectively to, for example, 10 out of 10.

In an embodiment, the controller 200 is configured to analyze the collected dataset. The analysis may be achieved using at least one algorithm that is adapted to determine a first plan (to be generated by the digital assistant) having the highest acceptance score, i.e., the highest probability to be accepted by the user and to fulfill the at least one predetermined goal. That is, the collected dataset may be fed into the at least one algorithm, therefore allowing to determine a first plan having the highest probability score to be accepted by the user and for causing fulfillment of the at least one predetermined goal.

As an example, the controller 200 may generate a suggestion (i.e., executing a plan) for the user to start an on-line yoga class as the user usually does, but the user refuses. According to the same example, the collected and analyzed dataset indicates that the user finished eating three hours ago, that the weather outside is great and the air pollution level is low. According to the same example, an alternative plan having the highest acceptance score to fulfill the at least one goal may be a plan that suggests the user to go out for a walk.

It should be noted that going out for a walk may replace the yoga class and allows the achievement of the goal of performing a daily workout (e.g., physical activity goal). In an embodiment, the analysis of the collected dataset may further include performing at least a first calculation of a probability of each of a plurality of possible plans to be accepted by the user and to fulfill the at least one predetermined goal. Then, a probability score may be determined for each of the plurality of possible plans based on the result of the first calculation. Thus, the controller 200 may be configured to select the plan having the highest acceptance score to fulfill the at least one predetermined goal.

According to the abovementioned example, after the user rejects the recommendation of doing yoga, the controller 200 may collect and analyze a dataset indicating that there is only a one-hour time window in the user's schedule (e.g., by searching in the user's electronic calendar), and therefore the controller 200 suggests the user to reschedule the on-line yoga class to that time window.

In an embodiment, the controller 200 is further configured to execute and present the determined first plan, having the highest acceptance score, to the user. Presenting (e.g., executing) a plan may be performed by using one or more resources (e.g., the resources 150) that are communicatively connected to and controlled by the digital assistant 120, such as speakers, a display unit, a smartphone that is communicatively connected to the digital assistant, and the like. For example, the controller 200 may determine that the plan having the highest acceptance score to be accepted by the user and to fulfill a certain predetermined goal, includes a suggestion to play a cognitive game. According to the same example, the suggestion (i.e., plan) may be presented to the user by emitting a sentence (using at least a speaker).

According to one embodiment, after the first plan is presented to the user, the controller 200 may be configured to collect at least a second feedback data from the user regarding the generated first plan. That is, after the first plan is generated and presented to the user, the controller 200 is configured to monitor the user's reaction to determine whether the user accepts or rejects the first plan (e.g., recommendation), the rejection intensity level, and so on.

According to another embodiment, upon determination that the user rejects the first plan, the controller 200 is further configured to perform at least a second calculation of the probability of a plurality of possible plans to be accepted by the user and to fulfill the predetermined goal. For example, the presented first plan, having the highest acceptance score to be accepted by the user and to fulfill at least one predetermined goal, includes a suggestion for the user to go out for a walk before dinner (to fulfill a goal of doing a daily physical activity). According to the same example, when the user rejects the suggestion (e.g., first plan) a second calculation is performed in which all other possible plans are considered (except the first plan of suggesting going out for a walk).

According to further embodiment, based on the results of the second calculation, the controller 200 is configured to determine a second plan having a second highest acceptance score to be accepted by the user and to fulfill the at least one predetermined goal. Then, the second plan is presented to the user. It should be noted that feedback data (e.g., a third feedback data) may be collected with respect to the presented second plan in order to determine the user's reaction regarding the presented second plan.

It should be further noted that in case the user rejects the second plan (e.g., a suggestion, recommendation) the controller 200 may perform a resolution process. The resolution process may include preventing other suggestions from being presented to the user (stop bothering the user), performing additional calculations of the probability of each of the plurality of possible plans to be accepted by the user and to fulfill the at least one predetermined goal to determine which of the plurality of plans has the third highest acceptance score, and so on.

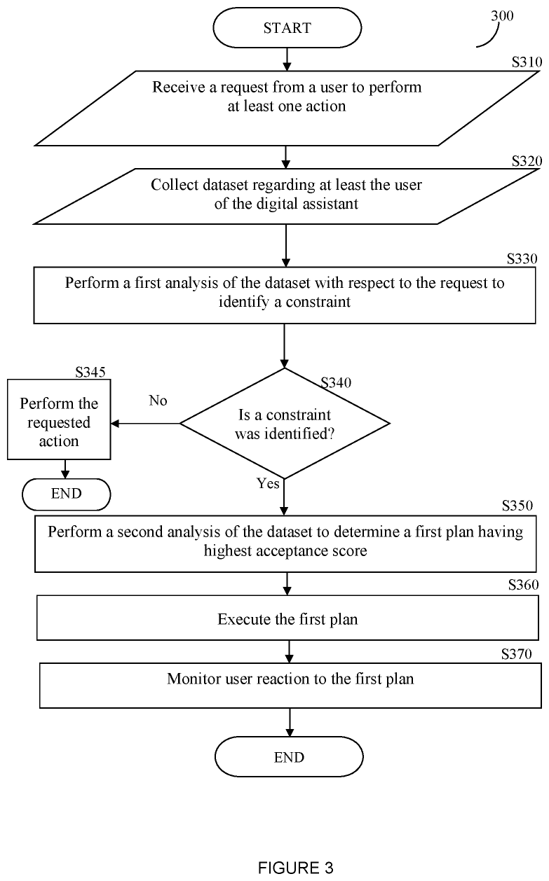

According to a further embodiment, and as further described herein below with respect to FIG. 3, the controller 200 may be configured to receive a request from the user of the digital assistant, to perform at least one action. For example, the request may indicate that the user wishes to listen to music, call a friend, dictate an electronic message to the digital assistant while driving, turn on the TV, and so on. The user request may be referred to as a temporary goal for the digital assistant 120. Thus, the controller 200 may be used for fulfilling the user's temporary goal given a certain limitation(s) as further described herein below. After the request is received, a dataset may be collected from one or more sources. A source is a source of information, such as for example, a user's electronic calendar, social media account, sensors, navigation program, database, and so on. The source may be realized in include software, hardware and a combination thereof. Further, an interface, such as API, may be provided to such source.

The dataset may include, for example, user's goals achievement status, data regarding the user's patterns and habits, user's schedule, calendar, real-time data, data that is gathered from the user's social media accounts, data that is gathered from websites and databases, and so on. The controller 200 may be configured to perform a first analysis of the collected dataset with respect to the request (e.g., the temporary goal).

A first algorithm, such as a machine learning algorithm, may be used for determining whether at least one constraint of the user constraints for fulfilling the user request (e.g., temporary goal) is identified. Upon determination that a limitation is identified, the controller 200 may be configured to perform a second analysis. The second analysis may include applying at least a second algorithm to the dataset. The second algorithm (e.g., a machine learning algorithm) may be used for determining a first plan, to be generated by the digital assistant, having the highest acceptance score to be accepted by the user.

According to another embodiment, the determined first plan may have the highest acceptance score to be accepted by the user and to fulfill at least one predetermined goal. A predetermined goal may be, for example, and without limitations, keeping the user safe, maintain physical health of the user, improve social relationship of the user, assist the user with managing the user's schedule, and so on. According to another embodiment, the first plan having the highest acceptance score is executed and presented to the user using one or more resources (e.g., the resources 150), as further described herein below with respect to FIG. 3.

As an example, the user asks the digital assistant 120 to start navigating to a certain destination (when the digital assistant operates at the user's electric vehicle). According to the same example, the user selects a certain route that is the shortest route to get to the destination. By collecting and analyzing a dataset, it is determined that in order to safely arrive to the destination the vehicle's battery must be recharged. The determination that the vehicle's battery must be recharged may be identified as a limitation or constraint with respect to the user's request. According to the same example, by applying the second algorithm to the collected dataset, it is determined that the plan having the highest acceptance score suggests the user to select an alternative route which is a longer route, but includes a charging station.

In an embodiment, the user may reject the suggested first plan. Such a rejection may be identified by the controller 200, as further discussed herein below with respect of FIG. 3. Upon determination that the user rejects the first plan, the controller 200 may be configured to perform a second computation of the probability of each of a plurality of possible plans to be accepted by the user and to fulfill the at least one predetermined goal. Thus, a second plan having the second-best probability to be accepted by the user and fulfill the predetermined goal may be determined.

FIG. 3 shows an example flowchart 300 of a method for customizing a plan for a user of a digital assistant in response to user's constraints, according to an embodiment. The method described herein may be executed by the controller 200 that is further described herein above with respect to FIG. 2.

At S310, a request is received from a user of the digital assistant to perform at least one action by the digital assistant. The action may be identified in the initial policy of the digital assistant. The request may be collected using one or more sensors (e.g., the sensors 140). As an example, the request may indicate that the user wishes to watch a movie added to the user's watching list yesterday.

At S320, a dataset is collected from one or more sources. Examples for potential sources are provided above. The collected dataset may include, for example, the user's goals achievement status, data regarding the user's patterns and habits, user's schedule, calendar, real-time data (of the user and the environment near the user) that may be sensed by one or more sensors (e.g., the sensors 140), data that is gathered from the user's social media account, data that is gathered from websites and/or databases (e.g., weather forecast), and so on.

At S330, a first analysis of the dataset is performed to identify at least one constraint with respect to the request. The first analysis may be achieved by applying at least a first algorithm to the dataset and the request. The first algorithm may be adapted to determine whether at least one limitation for fulfilling the user request is identified. That is, the collected dataset and the request (e.g., request data) may be fed into the first algorithm, therefore allowing to determine if a limitation for executing the user's request is identified.

As an example, the request may indicate that the user asks the digital assistant 120 to play a certain movie at a certain day and time. According to the same example, the collected dataset indicates that the length of the movie is 90 minutes, it is Sunday, the current time is 3 pm and that the user usually meets her/his friends at the park every Sunday at 3:30 pm for an hour. Therefore, the weekly meeting at the park (that may be retrieved form from the user's calendar) may be identified as a constraint (limitation). According to the same example, it may be determined that accepting the user's request, by playing the requested movie, may reduce the chances to fulfill a social relationship goal (e.g., of the week) as the user may probably miss the weekly meeting with her/his friends at the park. As another example, after the user requested the digital assistant 120 to turn off the lights in the house stating that the user is going to sleep, the controller 200 may collect data that indicates that the user has a scheduled meeting with a caregiver within 30 minutes at the user's home. That is, the meeting is a constraint with respect to the user request.

At S340, it is checked based on the result of the analysis, whether at least one constraint is identified, and if so, execution continues with S350; otherwise, execution continues with S345. At S345, upon determination that no constraint was identified, the at least one requested action is performed by the digital assistant 120.

At S350, a second analysis of the dataset is performed to determine a first plan with a highest acceptance score. The acceptance score is characterized with a score indicating high probability for acceptance by the user. Such a plan customizes the initial policy based on the identified constraints. Specifically, the second analysis may include applying at least a second algorithm to the dataset indicating the identified constraint(s). The second algorithm may be adapted to determine a first plan, thereby customizing the initial policy.

According to another embodiment, the determined first plan may have the highest acceptance score to be accepted by the user and to fulfill at least one predetermined goal. A predetermined goal may be, for example, keeping the user safe, maintain physical health of the user, improve social relationship of the user, assist the user with managing the user's schedule, and so on. In an embodiment, the predetermined goal may be a temporary goal set by the user, i.e., a user request. That is, the collected dataset may be fed into the second algorithm, therefore allowing to determine a first plan having the highest acceptance score. The first plan causes fulfillment of the at least one predetermined goal.

As an example, the user asks the digital assistant to listen to Jazz music. By collecting and analyzing the dataset it is determined that the time is 6 pm, the weather outside is good, that the user has been listening to music for more than 3 hours today and that a physical activity goal achievement status indicates that the user needs to do some sport to get closer to fulfill the daily physical activity goal. In this example, user's request to listen to music at 6 pm may reduce the chances to fulfill the daily physical activity goal, so the low achievement status of the physical activity may be identified as a limitation. According to the same example, by applying the second algorithm to the collected dataset, it is determined that the plan having the highest acceptance score by the user and to fulfill the predetermined goal is explaining to the user that she or he has been sitting on the couch for more than three hours and suggesting the user to go out for a 25 minutes' walk since it is a nice weather outside.

It should be noted that the second analysis of the dataset may include performing a first calculation of a probability of each of the possible plans to be accepted by the user and to fulfill the at least one predetermined goal. Then, an acceptance score for each of the plurality of possible plans may be determined based on the result of the first calculation. That is, there may be, for example, 10 optional plans that may contribute to the fulfillment of a certain predetermined goal and each of the plans may have a different acceptance score. Thus, based on the result of the calculation, one out of the ten plans is identified as the plan that has the highest acceptance score by the user and to fulfill the predetermined goal.

At S360, the determined first plan having the highest acceptance score is executed and presented to the user. Presenting, or executing, a plan may be performed using one or more resources (e.g., the resources 150) that are communicatively connected to and controlled by the digital assistant. Examples for resources include speakers, a display unit, a smartphone that is communicatively connected to the digital assistant, and so on.

For example, the controller (e.g., the controller 200) may determine that the plan having the highest acceptance score by the user and fulfills a certain predetermined goal includes a suggestion to play a cognitive game. According to the same example, the suggestion (i.e., plan) may be presented to the user by emitting a sentence (using at least a speaker).

In an embodiment, at S370, after the first plan is generated and presented to the user, the user's reaction may be monitored to determine whether the user accepts or rejects the first plan. To this end, feedback data is collected from the user regarding the executed first plan and the feedback data is analyzed. Based on the results of the analysis of the feedback data, and upon determination that the user rejected the first plan, a second calculation of a probability of each of a plurality of possible plans to be accepted by the user and fulfills the at least one predetermined goal, may be performed.

According to one embodiment, a second plan having a second highest acceptance score may be determined. Then, the second plan may be presented to the user. It should be noted that in case the user rejects the second plan, a resolution process may be executed. The resolution process may include preventing other suggestions from being presented to the user (stop bothering the user), performing additional calculations of the probability of each of the plurality of possible plans to be accepted by the user and fulfilling the at least one predetermined goal in order to determine which of the plurality of alternative plans has the highest acceptance score, and so on. In an embodiment, no second score is calculated, but rather a plan having the second-highest score is selected based on the analysis performed at S350.

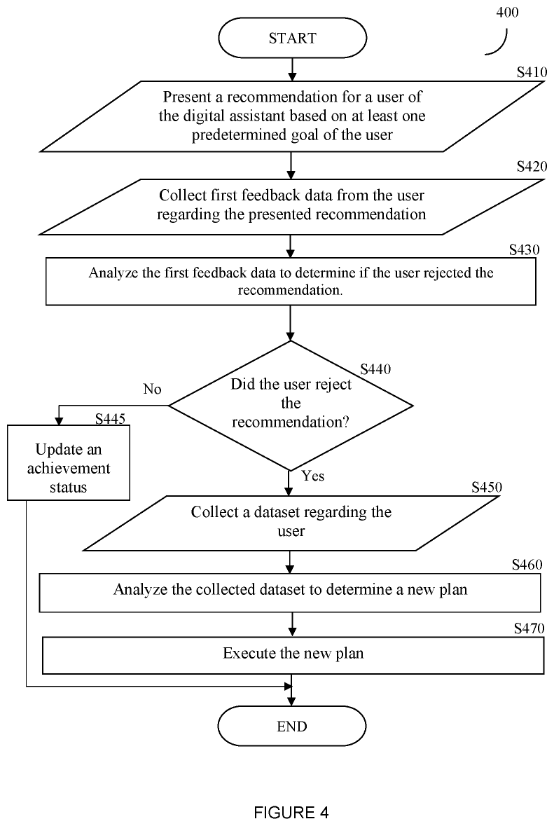

FIG. 4 shows an example flowchart 400 of a method for further customizing a policy in response to refusal of the user to accept a suggested plan, according to an embodiment. The method described herein may be performed by the controller 200 that is further described herein above with respect to FIG. 2.

At S410, a recommendation defined in an initial policy is presented to a user of a digital assistant based on at least one predetermined goal of the user. The recommendation may be previously determined and generated based on an achievement status of one of more goals.

At S420, a first feedback data is collected from the user with respect to the presented recommendation. The first feedback data may include real-time data that may be collected from sensors (e.g., the sensors 140) of the digital assistance.

At S430, the first feedback data is analyzed to determine if the user rejected the recommendation. The analysis of the first feedback data may be achieved using one or more computer vision techniques, audio signal processing techniques, machine learning techniques, and the like. For example, the user may respond to the presented recommendation by saying “not interested”. The user's voice command would be processed by an audio signal processing technique (e.g., speech recognition) to determine a rejection of the recommendation.

At S440, it is checked, based on the result of the analysis, whether the user rejected the recommendation and if so, execution continues with S450; otherwise, execution continues with S445. At S445, upon determination that the user accepted the recommendation and performed a corresponding action, an achievement status of at least one predetermined goal may be updated, respectively as further discussed herein above with respect to FIG. 2.

At S450, upon determination that the user rejects the recommendation (e.g., refuses to perform a suggested action that should contribute to fulfillment of a predetermined goal), a dataset is collected. The dataset may include for example, data regarding the user's patterns and habits, user's schedule, real-time data that may be sensed by the sensors (e.g., the sensors 140), data that is gathered from the user's social media accounts, data that is gathered from websites and/or databases (e.g., weather forecast), and so on.

At S460, the collected dataset is analyzed to determine a new plan. The new plan would customize the initial policy, so that future recommendation would be accepted by the user. The analysis may be achieved using at least one algorithm, such as a machine learning algorithm, that is trained to determine a plan (to be generated by the digital assistant) having a highest acceptance score. The score defines the probability that plan would be accepted and fulfill the at least one predetermined goal, as further discussed herein above with respect to FIGS. 2 and 3.

Examples for such determined plan may include, for example, a suggestion for the user to perform an action such as, go out for a walk, take medications now and not later since according to the user's schedule she or he will have company soon, and so on.

At S470, the new plan (e.g., a suggestion to perform an action) is executed and presented to the user. Execution of a plan may be performed using resources (e.g., the resources 150) as further discussed herein above with respect to FIG. 2.

It should be noted that after the determined plan is generated and presented to the user the user's reaction may be monitored to determine whether the user accepts or rejects such plan (e.g., recommendation), the rejection intensity level, and so on. According to one embodiment, upon determination that the user rejects the presented plan, a second plan having a second highest acceptance score to be accepted by the user and to fulfill the at least one predetermined goal may be determined. Then, the second plan may be presented to the user.

It should be noted that feedback data may be collected with respect to the presented second plan in order to determine the user's reaction regarding the presented second plan. It also should be noted that in case the user rejects the second plan, a resolution process may be executed. The resolution process may include preventing other suggestions from being presented to the user (stop bothering the user), perform additional calculation of the probability of each of the plurality of possible plans to be accepted by the user and to fulfill the at least one predetermined goal to determine which of the plurality of alternative plans has the highest probability score, and so on.

The various disclosed embodiments can be implemented as hardware, firmware, software, or any combination thereof. Moreover, the software is preferably implemented as an application program tangibly embodied on a program storage unit or computer readable medium. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such computer or processor is explicitly shown. In addition, various other peripheral units may be connected to the computer platform such as an additional data storage unit and a printing unit.

All examples and conditional language recited herein are intended for pedagogical purposes to aid the reader in understanding the principles of the disclosure and the concepts contributed by the inventor to furthering the art and are to be construed as being without limitation to such specifically recited examples and conditions. Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

A person skilled-in-the-art will readily note that other embodiments of the disclosure may be achieved without departing from the scope of the disclosed disclosure. All such embodiments are included herein. The scope of the disclosure should be limited solely by the claims thereto.

What is claimed is:

1. A method for generating a modified input/output (I/O) device policy in response to constraints of a user, comprising:
determining at least one constraint of a user using the I/O device policy in response to a request to perform a plan defined in the policy, wherein the at least one constraint is determined based on a first dataset collected from different electronic sources connected to the I/O device;
generating a first plan customizing the policy based on the at least one determined constraint, wherein the first plan demonstrates a high acceptance score, wherein the acceptance score defines a probability that the user accepts and fulfills the first plan; and
executing the first plan defined in the customized policy, wherein executing the first plan further includes causing the I/O device to activate an action that complies with the at least one determined constraint.

2. The method of claim 1, further comprising:
monitoring a feedback of a user to the executed first plan;
generating a second plan further customizing the policy when the user rejects the first plan; and
executing the second plan.

3. The method of claim 2, wherein generating the second plan further comprises:
collecting a second dataset from a plurality or resources; and
analyzing the second dataset to determine an alternative plan to the first plan having a high acceptance score.

4. The method of claim 3, wherein collecting the first dataset and the second dataset further comprising:
collecting, using a plurality of sensors connected to the I/O device, at least real-time data on the user and an environment in a predetermined proximity to the user.

5. The method of claim 4, wherein the first dataset and the second dataset includes historical data and routine information related to activities of the user.

6. The method of claim 1, wherein generating the first plan customizing the policy further comprising:
generating the first plan based on at least one goal defined for the user.

7. The method of claim 6, further comprising:
analyzing the first dataset to generate at least a plurality of plans that comply with the at least one constraint and the at least goal;
computing for each of the plurality of plans the probability of acceptance of the respective plan by the user; and
selecting the plan with the highest probability, wherein the highest probability of the plan is the high acceptance score.

8. The method of claim 7, wherein the analysis of the first dataset and the computation of the probabilities of acceptance is performed using a machine learning model.

9. The method of claim 1, wherein execution of any of the first plan and the second plan further comprising:
performing at least an action defined in the respective plan by a digital assistant included in the I/O device.

10. A non-transitory computer readable medium having stored thereon instructions for causing a processing circuitry to execute a process, the process comprising:
determining at least one constraint of a user using the I/O device policy in response to a request to perform a plan defined in the policy, wherein the at least one constraint is determined based on a first dataset collected from different electronic sources connected to the I/O device;
generating a first plan customizing the policy based on the at least one determined constraint, wherein the first plan demonstrates a high acceptance score, wherein the acceptance score defines a probability that the user accepts and fulfills the first plan; and
executing the first plan defined in the customized policy, wherein executing the first plan further includes causing the I/O device to activate an action that complies with the at least one determined constraint.

11. A system for generating a modified input/output (I/O) device policy in response to constraints of a user, comprising:
a processing circuitry; and
a memory, the memory containing instructions that, when executed by the processing circuitry, configure the system to:
determine at least one constraint of a user using the I/O device policy in response to a request to perform a plan defined in the policy, wherein the at least one constraint is determined based on a first dataset collected from different electronic sources connected to the I/O device;
generate a first plan customizing the policy based on the at least one determined constraint, wherein the first plan demonstrates a high acceptance score, wherein the acceptance score defines a probability that the user accepts and fulfills the first plan; and
execute the first plan defined in the customized policy, wherein executing the first plan further includes causing the I/O device to activate an action that complies with the at least one determined constraint.

12. The system of claim 11, wherein the system is further configured to:
monitor a feedback of a user to the executed first plan;
generate a second plan further customizing the policy when the user rejects the first plan; and
execute the second plan.

13. The system of claim 12, wherein the system is further configured to:
collect a second dataset from a plurality or resources; and
analyze the second dataset to determine an alternative plan to the first plan having a high acceptance score.

14. The system of claim 13, wherein the system is further configured to:
collect, using a plurality of sensors connected to the I/O device, at least real-time data on the user and an environment in a predetermined proximity to the user.

15. The system of claim 14, wherein the first dataset and the second dataset include historical data and routine information related to activities of the user.

16. The system of claim 13, wherein the system is further configured to:
generate the first plan based on at least one goal defined for the user.

17. The system of claim 16, wherein the system is further configured to:
analyze the first dataset to generate at least a plurality of plans that comply with the at least one constraint and the at least goal;
compute for each of the plurality of plans the probability of acceptance of the respective plan by the user; and
select the plan with the highest probability, wherein the highest probability of the plan is the high acceptance score.

18. The system of claim 17, wherein the analysis of the first dataset and the computation of the probabilities of acceptance is performed using a machine learning model.

19. The system of claim 13, wherein the system is further configured to:
perform at least an action defined in the respective plan by a digital assistant included in the I/O device.
